(19) United States
(12) Patent Application Publication
(10) Pub. No.: US 2019/0320986 A1
(43) Pub. Date: Oct. 24, 2019

(54) HEART RATE ADAPTIVE FILTERING DEVICE, PHYSIOLOGY INFORMATION SENSING DEVICE AND METHOD FOR PROCESSING PULSATILE SIGNAL

(71) Applicant: GIN-CHUNG WANG, LINCOLNSHIRE, IL (US)

(72) Inventor: GIN-CHUNG WANG, LINCOLNSHIRE, IL (US)

(21) Appl. No.: 15/960,600

(22) Filed: Apr. 24, 2018

(51) Int. Cl. A61B 5/00; A61B 5/021

(52) U.S. Cl. CPC A61B 5/725; A61B 5/02416; A61B 5/02116

(57) ABSTRACT

A heart rate adaptive filter used in a physiology information sensing device is illustrated. The heart rate adaptive filter has a heart rate measuring device and a frequency configurable filter electrically connected to the heart rate measuring device. The heart rate measuring device can determine a heart rate or a heart rate signal according to a pulsatile signal sensed from an individual user. The frequency configurable filter can determine a transfer function thereof according to the heart rate or the heart rate signal and apply the transfer function to the pulsatile signal to generate an adaptively filtered pulsatile signal. Since the transfer function is determined according to the heart rate or the heart rate signal which has information of a characteristic frequency of the pulsatile signal, the signal-to-noise ratio of the adaptively filtered pulsatile signal can have improvement.

### TECHNICAL FIELD

The present disclosure relates to a physiology information sensing device, and in particular to a heart rate adaptive filtering device used in a physiology information sensing device, the physiology information sensing device and a method for processing a pulsatile signal.

### RELATED ART

The pulsatile signal is originated form a heart, such as ECG (electrocardiography), PPG (photoplethysmography) and blood pressure signal. Specifically, the pulsatile signal is driven by heartbeat. Nowadays, only very few indicators of physiology information can be derived from waveforms, such as a heart rate, a heart rate variability, a diastolic blood pressure and a systolic blood pressure.

The pulsatile signal plays an important role in the function of determining a heart rate. However, the conventional filter for processing the pulsatile signal has a fixed bandwidth, which may cause the incorrect indicators determined or calculated based on the pulsatile signal since pulsatile signals of individual users may have different characteristic frequencies.

### SUMMARY

An objective of the present disclosure is to provide a heart rate adaptive filtering device used in a physiology information sensing device, which has at least one adjustable parameter being varied according to a heart rate associated with a pulsatile signal sensed from an individual user.

Another objective of the present disclosure is to provide the physiology information sensing device using the heart rate adaptive filtering device to filter the pulsatile signal.

Another objective of the present disclosure is to provide a method for processing the pulsatile signal dedicated to determine one or more indication signals of physiology information associated with the individual user.

To achieve at least one of the above objectives, the present disclosure provides a heart rate adaptive filter. The heart rate adaptive filter has a heart rate measuring device and a frequency configurable filter electrically connected to the heart rate measuring device. The heart rate measuring device can determine a heart rate or a heart rate signal according to a pulsatile signal sensed from an individual user. The frequency configurable filter can determine a transfer function thereof according to the heart rate or the heart rate signal and apply the transfer function to the pulsatile signal to generate an adaptively filtered pulsatile signal.

In an embodiment of the present disclosure, the heart rate adaptive filter further comprises a processing device electrically connected between the heart measuring device and the frequency configurable filter. The processing device can determine a filter controlling signal according to the heart rate or the heart rate signal, and transmit the filter controlling signal to the frequency configurable filter. The frequency configurable filter can determine the transfer function according to the filter controlling signal and filter the pulsatile signal to generate an adaptively filtered pulsatile signal by using the transfer function.

In an embodiment of the present disclosure, the pulsatile signal, the filter controlling signal and the frequency configurable filter are analog.

In an embodiment of the present disclosure, when the pulsatile signal, the filter controlling signal and the frequency configurable filter are analog, the frequency configurable filter comprises at least one adjustable impedance component.

In an embodiment of the present disclosure, the adjustable impedance component comprises a variable resistor, a variable capacitor, a variable inductor or a switching capacitor circuit.

In an embodiment of the present disclosure, the heart rate adaptive filter further comprises an analog-to-digital converter electrically connected to the heart measuring device. The analog-to-digital converter can convert the pulsatile signal to a digital pulsatile signal. The heart measuring device can determine the heart rate or the heart rate signal according to the digital pulsatile signal, and the frequency configurable filter is a configurable digital filter which uses the transfer function to filter the digital pulsatile signal to generate the adaptively filtered pulsatile signal.

In an embodiment of the present disclosure, a sampling rate of the analog-to-digital converter is adjusted according to the heart rate or the heart rate signal

In an embodiment of the present disclosure, when the frequency configurable filter is the configurable digital filter, the heart rate measuring device comprises a digital filter electrically connected to the analog-to-digital converter, a comparator electrically connected to the digital filter and a counter electrically connected to the comparator and the configurable digital filter. The digital filter can filter the digital pulsatile signal to generate a digital filtered pulsatile signal. The comparator can compare the digital pulsatile signal and a threshold voltage to generate a comparison signal. The counter can determine the heart rate or the heart rate signal according to the comparison signal.

In an embodiment of the present disclosure, when the frequency configurable filter is the configurable digital filter, and the heart rate measuring device comprises the digital filter, the comparator and the counter, the heart rate adaptive filter can be a digital processing circuit or chip.

To achieve at least one of the above objectives, the present disclosure provides a physiology information sensing device. The physiology information sensing device comprises a pulsatile signal sensing device, a heart rate adaptive filtering device, electrically connected to the pulsatile signal sensing device and an indicator generating device electrically connected to the heart rate adaptive filtering device. The pulsatile signal sensing device can sense a pulsatile signal from an individual user. The heart rate adaptive filtering device can determine a heart rate or a heart rate signal according to the pulsatile signal, determine a transfer function thereof according to the heart rate or the heart rate signal, and apply the transfer function to the pulsatile signal to generate an adaptively filtered pulsatile signal. The indicator generating device can generate at least one indication signal according to the adaptively filtered pulsatile signal, wherein the indication signal is an indicator for evaluating physiology status of the individual user.

In an embodiment of the present disclosure, the heart rate adaptive filtering device can be carried out by one of the above implementations.

In an embodiment of the present disclosure, the pulsatile sensing device is a blood pressure sensor, an assembly of an optical sensor, an electrode assembly or an image array device, and the indicator generating device is a peak value acquiring device for acquiring a maximum value and a minimum value of the adaptively filtered pulsatile signal, a phase analysis device for acquiring a phase of the adaptively filtered pulsatile signal or an absolute value circuit for acquiring an amplitude of the adaptively filtered pulsatile signal.

To achieve at least one of the above objectives, the present disclosure provides a method for processing a pulsatile signal sensed from an individual user, which comprises steps of: determining a heart rate or a heart rate signal according to the pulsatile signal; determining a transfer function thereof according to the heart rate or the heart rate signal; and applying the transfer function to the pulsatile signal to generate an adaptively filtered pulsatile signal.

To sum up, the present disclosure provides a heart rate adaptive filtering device and a method for processing a pulsatile signal, which have an improved signal-to-noise ratio of an adaptively filtered pulsatile signal and enhance accuracy of an indication signal generated from the adaptively filtered pulsatile signal, thus the physiology information sensing device using such device and method can accurately evaluate physiology status of the individual user.

### BRIEF DESCRIPTION OF THE DRAWINGS

In order that the present disclosure may be better understood and readily carried into effect, certain embodiments of the present disclosure will now be described with reference to the accompanying drawings, wherein:

FIG. 1 is a block diagram of a physiology information sensing device according to an embodiment of the present disclosure;

FIG. 2 is a block diagram of a heart rate adaptive filtering device according to an embodiment of the present disclosure; and

FIG. 3 is a flow chart of a method for processing a pulsatile signal sensed from an individual user according to one embodiment of the present disclosure.

### DETAILED DESCRIPTION OF THE PREFERRED EMBODIMENTS

To make it easier for the examiner to understand the objects, characteristics and effects of this present disclosure, embodiments together with the attached drawings for the detailed description of the present disclosure are provided.

An embodiment of the present disclosure provides a heart rate adaptive filtering device having at least one adjustable parameter, wherein the adjustable parameter is varied according to a heart rate signal (or a heart rate) which is determined or calculated based on a pulsatile signal sensed from an individual user, and the adjustable parameter can be a coefficient of a transfer function for example. The heart rate adaptive filtering device is used in a physiology information sensing device for filtering the pulsatile signal to generate an adaptively filtered pulsatile signal, and then an indicator generating device in the physiology information sensing device determines or calculates at least one indication signal according to the adaptively filtered pulsatile signal, wherein the indication signal is an indicator for evaluating physiology status of the individual user.

The heart rate adaptive filtering device comprises a heart rate measuring device and a frequency configurable filter, wherein the heart rate measuring device is electrically connected to the heart rate measuring device. The heart rate measuring device is used to determine or calculate a heart rate signal (or heart rate) according to the pulsatile signal, wherein the pulsatile signal can be analog or digital, and the present disclosure is not limited thereto. The frequency configurable filter has a transfer function for filtering the pulsatile signal, wherein the transfer function defining a bandwidth, a gain and a center frequency of the frequency configurable filter can be determined according to the adjustable parameter of the frequency configurable filter.

Since pulsatile signals of individual users may have different characteristic frequencies, and heart rates have information of the characteristic frequencies, the heart rate signal (or heart rate) can be used to configure the frequency configurable filter. Therefore, the adjustable parameter of the frequency configurable filter is varied according to the heart rate signal (or heart rate) determined or calculated by the heart rate measuring device, the transfer function can be varied according to the characteristic frequency of the pulsatile signal of the individual user, and adaptive filtering of the frequency configurable filter can be applied to the pulsatile signal of the individual user.

In an embodiment of the present disclosure, a processing device can be disposed between the heart rate measuring device and the frequency configurable filter, and the processing device receives the heart rate signal (or heart rate) and generates a filter controlling signal to the frequency configurable filter according to the heart rate signal (or heart rate), wherein the transfer function of the frequency configurable filter is determined according to the filter controlling signal.

In another embodiment of the present disclosure, the heart rate signal (or heart rate) is directly sent to the frequency configurable filter to determine be transfer function of the frequency configurable filter. The frequency configurable filter can be analog or digital, and the present disclosure is not limited thereto.

Moreover, according to the above concept of the present disclosure, an embodiment of the present disclosure provides a method for processing the pulsatile signal sensed from the individual user, wherein the method can be used in the heart rate adaptive filtering device of the physiology information sensing device, but the application of the method is not limited to the physiology information sensing device.

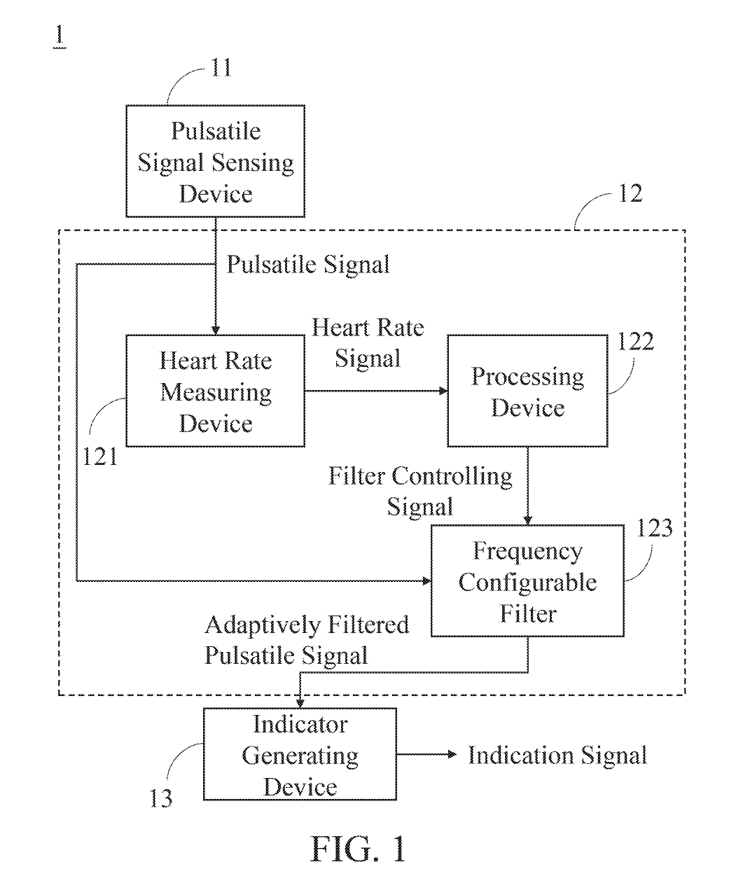

Referring to FIG. 1, FIG. 1 is a block diagram of a physiology information sensing device according to an embodiment of the present disclosure. The physiology information sensing device 1 comprises a pulsatile signal sensing device 11, a heart rate adaptive filtering device 12 and an indicator generating device 13, wherein the pulsatile signal sensing device 11 is electrically connected to the heart rate adaptive filtering device 12, and the heart rate adaptive filtering device 12 is electrically connected to the indicator generating device 13.

The pulsatile signal sensing device 11 can be a blood pressure sensor or an assembly of an optical sensor (such as a photodiode) and a light source (such as a light emission diode (LED) or a laser), which can acquire the pulsatile signal from an individual user. The above implementation of the pulsatile sensing device 11 is not used to limit the present disclosure, and the type of the pulsatile signal can be dependent on the implementation of the pulsatile sensing device 11, which may be an ECG signal, a PPG signal or a blood pressure signal. For example, the pulsatile signal sensing device 11 may be an electrode assembly or an image array device.

The heart rate adaptive filtering device receives the pulsatile signal from the pulsatile signal sensing device 11 and uses its transfer function to filter (or process) the pulsatile signal. The heart rate adaptive filtering device 12 can determine or calculate a heart rate signal (or heart rate) according to the pulsatile signal, and then uses the heart rate signal (or heart rate) to determines its transfer function to make a characteristic frequency of the transfer function correspond to a characteristic frequency of the pulsatile signal of the individual user. Thus, adaptive filtering is applied to the pulsatile signal, and an adaptively filtered pulsatile signal is then transmitted to the indicator generating device 13. Since the characteristic frequency of the transfer function corresponds to the characteristic frequency of the pulsatile signal of the individual user, the adaptive filtering can improve the signal-to-noise ratio (SNR) of the adaptively filtered pulsatile signal.

The indicator generating device 13 is used to receive the adaptively filtered pulsatile signal and generates at least one indication signal according to the adaptively filtered pulsatile signal, wherein the indication signal functions as an indicator for evaluating physiology status of the individual user. The indicator generating device 13 may be a peak value acquiring device for acquiring a maximum value and a minimum value of the adaptively filtered pulsatile signal, a phase analysis device for acquiring a phase of the adaptively filtered pulsatile signal or an absolute value circuit for acquiring an amplitude of the adaptively filtered pulsatile signal, but the present disclosure is not limited thereto.

Still referring to FIG. 1, the heart rate adaptive filtering device 12 in the embodiment of FIG. 1 may be implemented as follows, but the present disclosure is not limited thereto. The heart rate adaptive filtering device 12 comprises a heart rate measuring device 121, a processing device 122 and a frequency configurable filter 123, wherein the processing device 122 is electrically connected to the heart rate measuring device 121 and the frequency configurable filter 123, the heart rate measuring device 121 is electrically connected to the pulsatile signal sensing device 11, and the frequency configurable filter 123 is electrically connected to the pulsatile signal sensing device 11 and the indicator generating device. In the embodiment, the pulsatile signal, the heart rate measuring device 121 and the frequency configurable filter 123 are analog or digital, but the present disclosure is not limited thereto.

The heart rate measuring device 121 can receive the pulsatile signal and determine or calculate the heart rate signal (or heart rate) according to the pulsatile signal. The processing device 122 can receive the heart rate signal (or heart rate) and determine a filtering control signal according to the heart rate signal (or heart rate), wherein the filtering control signal can be analog as well as the frequency configurable filter 123 and the pulsatile signal, but the present disclosure is not limited thereto. The frequency configurable filter 123 can receive the pulsatile signal and the filtering control signal. The frequency configurable filter 123 can further determine the transfer function thereof according to the filter controlling signal by varying at least one adjustable parameter thereof, and then filter the pulsatile signal by using the transfer function thereof to generate the adaptively filtered pulsatile signal.

By the way, when the frequency configurable filter 123 is analog, the frequency configurable filter 123 can have at least one variable impedance component, such as a variable resistor, a variable capacitor, a variable inductor or a switching capacitor circuit. Thus, by controlling the impedance of the frequency configurable filter 123, the time constant (such as RC time constant) of the frequency configurable filter 123 can be adjusted to vary the transfer function of the frequency configurable filter 123.

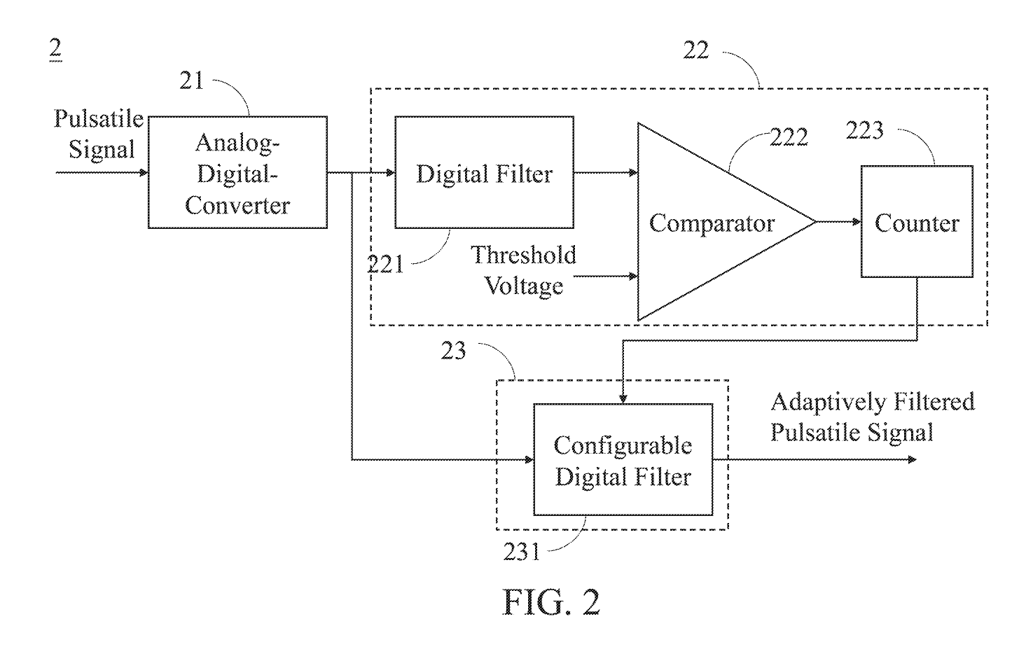

Another implementation of the heart rate adaptive filtering device is illustrated as follows. Referring to FIG. 2, FIG. 2 is a block diagram of a heart rate adaptive filtering device according to an embodiment of the present disclosure. The heart rate adaptive filtering device 2 comprises an analog-digital-converter 21, a heart rate measuring device 22 and a frequency configurable filter 23, wherein the heart rate measuring device 22 is electrically connected to the analog-digital-converter 21 and the frequency configurable filter 23, and the frequency configurable filter 23 is electrically connected to the analog-digital-converter 21. Compared to the embodiment of FIG. 1, the heart rate adaptive filtering device 2 further comprises the analog-digital-converter 21 but does not comprises the processing device.

The heart rate measuring device 22 can be implemented to comprise a digital filter 221, a comparator 222 and a counter 223. Further, the frequency configurable filter 23 can be implemented to comprise a configurable digital filter 231. The digital filter 221 is electrically connected to the analog-digital-converter 21 and the comparator 222, the counter 223 is electrically connected to the configurable digital filter 231 and the comparator 222, and the configurable digital filter 231 is electrically connected to the analog-digital-converter 21.

The pulsatile signal is analog, and the analog-digital-converter 21 can receive the pulsatile signal and convert the pulsatile signal to be digital. The digital pulsatile signal is transmitted to the digital filter 221. The digital filter 221 may have a bandwidth (P.S. the bandwidth may be fixed, but the present disclosure is not limited thereto) and filter out the components of the pulsatile signal outside the bandwidth.

The comparator 222 can receive the filtered pulsatile signal from the digital filter 221 and a threshold voltage, and further compare the filtered pulsatile signal and a threshold voltage to generate a comparison signal to the counter 223. The counter 223 can be a positive edge trigger counter, receive the comparison signal and count the heart rate according to the comparison signal.

The heart rate signal can be generated by the counter 223 and received by the configurable digital filter 231. The configurable digital filter 231 can determine the transfer function thereof according to heart rate signal, and the use the transfer function thereof to filter the digital pulsatile signal to generate the adaptively filtered pulsatile signal.

By the way, according to the above descriptions, the heart rate adaptive filtering device 2 can be implemented in a general purposed digital processing circuit or chip, but the present disclosure is not limited thereto. Furthermore, a sampling rate of the analog-digital-converter 21 can be adjustable, and the sampling rate can be determined according to the heart rate signal (or heart rate), so as to increase the accuracy of the indication signal generated from the adaptively filtered pulsatile signal.

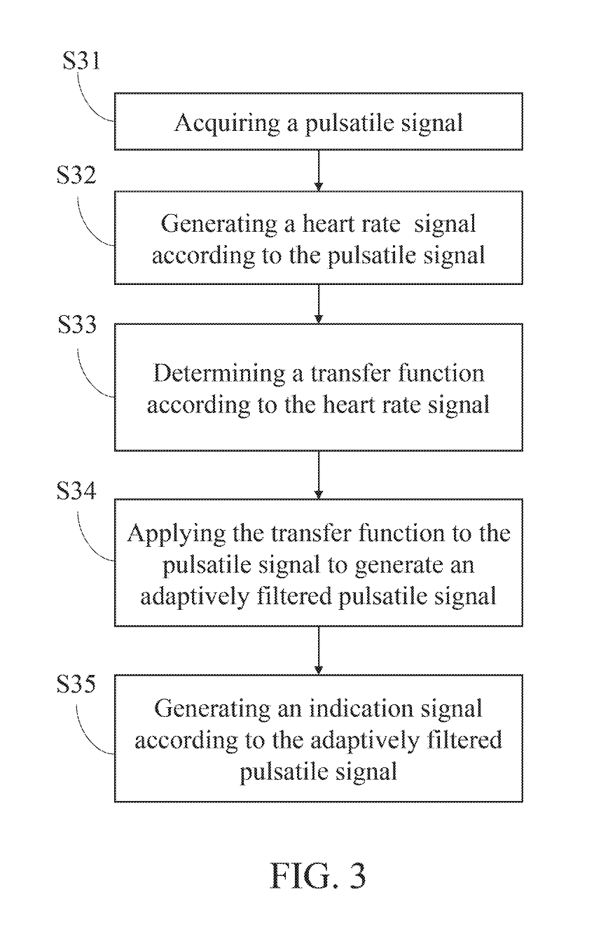

Further, a method for processing a pulsatile signal sensed from an individual user used in the heart rate adaptively filtering device is accordingly provided by the present disclosure. Referring to FIG. 3, FIG. 3 is a flow chart of a method for processing a pulsatile signal sensed from an individual user according to one embodiment of the present disclosure. Instead of the implementation of hardware components, the method can be implemented by software codes executed by a computing device. That is, the implementation of the method is not used to limit the present disclosure.

At step S31, a pulsatile signal is acquired by using a pulsatile signal sensing device to sense the body of the individual user. Then, at step S32, a heart rate signal is generated according to the pulsatile signal by using the heart rate measuring device, for example, the heart rate measuring device in FIG. 1 or FIG. 2, but the present disclosure is not limited thereto. For example, a frequency domain transformer (such as, FFT) can be used at step S32 to transform the pulsatile signal to the frequency domain pulsatile signal, and the center frequency (i.e. characteristic frequency) of the frequency domain pulsatile signal is acquired as the heart rate.

At step S33, a transfer function of a frequency configurable filter is determined according to the heart rate signal. Next, at step S34, the transfer function is applied to the pulsatile signal to generate an adaptively pulsatile signal. Further, at step S35, an indication signal is generated according to the adaptively pulsatile signal.

In conclusion, the present disclosure provides a heart rate adaptive filtering device and a method for processing a pulsatile signal. In such device or method, the pulsatile signal is firstly used to determine a heart rate, and the heart rate is then used to determine a transfer function to be applied to the pulsatile signal. Since the heart rate has the information of the characteristic frequency of the pulsatile signal, and the transfer function is determined according to the heart rate, an adaptively filtered pulsatile signal generated from such device or method has improvement in SNR, and accuracy of an indication signal generated from the adaptively filtered pulsatile signal is enhanced. Moreover, a physiology information sensing device using such device and method can accurately evaluate physiology status of the individual user.

While the present disclosure has been described by means of specific embodiments, numerous modifications and variations could be made thereto by those skilled in the art without departing from the scope and spirit of the present disclosure set forth in the claims.

What is claimed is:

1. A heart rate adaptive filter, comprising:
a heart rate measuring device, used to determine a heart rate or a heart rate signal according to a pulsatile signal sensed from an individual user; and
a frequency configurable filter, electrically connected to the heart rate measuring device, used to determine a transfer function thereof according to the heart rate or the heart rate signal, and apply the transfer function to the pulsatile signal to generate an adaptively filtered pulsatile signal.

2. The heart rate adaptive filter according to claim 1, further comprising:
a processing device, electrically connected between the heart measuring device and the frequency configurable filter, used to determine a filter controlling signal according to the heart rate or the heart rate signal, and transmit the filter controlling signal to the frequency configurable filter;
wherein the frequency configurable filter determines the transfer function according to the filter controlling signal and filters the pulsatile signal to generate an adaptively filtered pulsatile signal by using the transfer function.

3. The heart rate adaptive filter according to claim 2, wherein the pulsatile signal, the filter controlling signal and the frequency configurable filter are analog.

4. The heart rate adaptive filter according to claim 3, wherein the frequency configurable filter comprises at least one adjustable impedance component.

5. The heart rate adaptive filter according to claim 4, wherein the adjustable impedance component comprises a variable resistor, a variable capacitor, a variable inductor or a switching capacitor circuit.

6. The heart rate adaptive filter according to claim 1, further comprising:
an analog-to-digital converter, electrically connected to the heart measuring device, used to convert the pulsatile signal to a digital pulsatile signal;
wherein the heart measuring device determines the heart rate or the heart rate signal according to the digital pulsatile signal, and the frequency configurable filter is a configurable digital filter which uses the transfer function to filter the digital pulsatile signal to generate the adaptively filtered pulsatile signal.

7. The heart rate adaptive filter according to claim 6, wherein a sampling rate of the analog-to-digital converter is adjusted according to the heart rate or the heart rate signal.

8. The heart rate adaptive filter according to claim 6, wherein the heart rate measuring device comprises:
a digital filter, electrically connected to the analog-to-digital converter, used to filter the digital pulsatile signal to generate a digital filtered pulsatile signal;
a comparator, electrically connected to the digital filter, used to compare the digital pulsatile signal and a threshold voltage to generate a comparison signal; and
a counter, electrically connected to the comparator and the configurable digital filter, used to determine the heart rate or the heart rate signal according to the comparison signal.

9. The heart rate adaptive filter according to claim 8, wherein the heart rate adaptive filter is a digital processing circuit or chip.

10. A physiology information sensing device, comprising:
a pulsatile signal sensing device, used to sense a pulsatile signal from an individual user;
a heart rate adaptive filtering device, electrically connected to the pulsatile signal sensing device, used to determine a heart rate or a heart rate signal according to the pulsatile signal, determine a transfer function thereof according to the heart rate or the heart rate signal, and apply the transfer function to the pulsatile signal to generate an adaptively filtered pulsatile signal; and
an indicator generating device, electrically connected to the heart rate adaptive filtering device, used to generate at least one indication signal according to the adaptively filtered pulsatile signal, wherein the indication signal is an indicator for evaluating physiology status of the individual user.

11. The physiology information sensing device according to claim 10, wherein the heart rate adaptive filter comprises:
a heart rate measuring device, used to determine the heart rate or the heart rate signal according to the pulsatile signal; and
a frequency configurable filter, electrically connected to the heart rate measuring device, used to determine the transfer function according to the heart rate or the heart rate signal, and apply the transfer function to the pulsatile signal to generate the adaptively filtered pulsatile signal.

12. The physiology information sensing device according to claim 11, wherein the heart rate adaptive filter further comprises:
a processing device, electrically connected between the heart measuring device and the frequency configurable filter, used to determine a filter controlling signal according to the heart rate or the heart rate signal, and transmit the filter controlling signal to the frequency configurable filter;
wherein the frequency configurable filter determines the transfer function according to the filter controlling signal and filters the pulsatile signal to generate an adaptively filtered pulsatile signal by using the transfer function.

13. The physiology information sensing device according to claim 12, wherein the pulsatile signal, the filter controlling signal and the frequency configurable filter are analog.

14. The physiology information sensing device according to claim 13, wherein the frequency configurable filter comprises at least one adjustable impedance component comprising a variable resistor, a variable capacitor, a variable inductor or a switching capacitor circuit.

15. The physiology information sensing device according to claim 10, wherein the heart rate adaptive filter further comprises:
an analog-to-digital converter, electrically connected to the heart measuring device, used to convert the pulsatile signal to a digital pulsatile signal;
wherein the heart measuring device determines the heart rate or the heart rate signal according to the digital pulsatile signal, and the frequency configurable filter is a configurable digital filter which uses the transfer function to filter the digital pulsatile signal to generate the adaptively filtered pulsatile signal.

16. The physiology information sensing device according to claim 15, wherein a sampling rate of the analog-to-digital converter is adjusted according to the heart rate or the heart rate signal.

17. The physiology information sensing device according to claim 15, wherein the heart rate measuring device comprises:
a digital filter, electrically connected to the analog-to-digital converter, used to filter the digital pulsatile signal to generate a digital filtered pulsatile signal;
a comparator, electrically connected to the digital filter, used to compare the digital pulsatile signal and a threshold voltage to generate a comparison signal; and
a counter, electrically connected to the comparator and the configurable digital filter, used to determine the heart rate or the heart rate signal according to the comparison signal.

18. The physiology information sensing device according to claim 17, wherein the heart rate adaptive filter is a digital processing circuit or chip.

19. The physiology information sensing device according to claim 10, wherein the pulsatile sensing device is a blood pressure sensor, an assembly of an optical sensor, an electrode assembly or an image array device, and the indicator generating device is a peak value acquiring device for acquiring a maximum value and a minimum value of the adaptively filtered pulsatile signal, a phase analysis device for acquiring a phase of the adaptively filtered pulsatile signal or an absolute value circuit for acquiring an amplitude of the adaptively filtered pulsatile signal.

20. A method for processing a pulsatile signal sensed from an individual user, comprising steps of:
determining a heart rate or a heart rate signal according to the pulsatile signal;
determining a transfer function thereof according to the heart rate or the heart rate signal; and
applying the transfer function to the pulsatile signal to generate an adaptively filtered pulsatile signal.
